(19) United States
(12) Patent Application Publication
(10) Pub. No.: US 2006/0206210 A1
(43) Pub. Date: Sep. 14, 2006

(54) Artificial joint

(75) Inventors: Christian Abicht, Jena (DE); Peter Adam, Dachau (DE); Dietmar Kubein-Meesenburg, Goettingen (DE); Hans Naegerl, Gleichen (DE)

Correspondence Address: DAVIDSON, DAVIDSON & KAPPEL, LLC, 485 SEVENTH AVENUE, 14TH FLOOR, NEW YORK, NY, 10018, US

(73) Assignee: HJS GELENK SYSTEM GMBH, Muenchen (DE)

(21) Appl. No.: 10/537,969

(22) PCT Filed: Dec. 9, 2003
(86) PCT No.: PCT/DE03/04025

§ 371 Date: Feb. 10, 2006

(30) Foreign Application Priority Data
Dec. 10, 2002 (DE) 102 57 774.9

(51) Int. Cl. A61F 2/30; A61F 2/38

(52) U.S. Cl. 623/18.11; 623/20.32; 623/901

(57) ABSTRACT

An artificial joint for a patient includes a joint plateau having a recess including a first dimension and a joint overlay having a projection corresponding to the recess and a second dimension, the second dimension being larger than the first dimension at a body temperature of the patient. The projection is insertable into the recess by virtue of a temperature difference of at least one of the projection and the recess with respect to the body temperature. A contact area is formed between the projection and the recess at the body temperature. At least one of the second dimension and the contact area is determined such that fixation forces exerted on the projection at the body temperature create a state of stress in at least one of the joint overlay and the joint plateau, the state of stress improving at least one of a load-bearing capacity and a durability of the artificial joint.

The invention relates to an artificial joint with a joint plateau and a joint overlay which are connected to each other by means of a contact area defined by a recess and a projection inserted therein, whereby the projection has an overdimension with respect to the recess when it is at the body temperature of the patient and it can be inserted into the recess by virtue of a temperature difference of the projection or of the recess in comparison to the body temperature of the patient. Furthermore, the invention relates to a method for establishing a connection in such an artificial joint.

Such an artificial joint is already known, for example, from U.S. Pat. No. 4,878,916. There, the artificial joint has a rotation-symmetrical joint plateau and a socket-shaped joint overlay, which are connected to each other by means of a shrinkage connection. The joint overlay has an overdimension in the joint plateau at the body temperature of the patient, as a result of which it can only be inserted into the recess by virtue of a temperature difference in comparison to the body temperature of the patient.

Artificial joints of this generic type are also known from U.S. Pat. No. 5,858,020 and French Pat. Appl. No. 24 93 139 A1.

It has been found to be a drawback of the prior-art joints of the above-mentioned type that the load-bearing capacity of the artificial joint cannot be ensured on a permanent basis. In particular, damage to the joint parts cannot be reliably ruled out.

Furthermore, European Patent Application EP 04 95 340 A1 also discloses a modular assembly for the tibia segment of a knee-joint prosthesis that consists of a tibia plateau having a journal and of a joint overlay made of polyethylene. Stems of varying sizes can be inserted into the joint plateau, while joint overlays of different heights are held on all sides in a plane of the tibia plateau. In order to affix the joint overlay, the latter is introduced into the plane of the tibia plateau by means of a pivoting movement. However, it has been found to be disadvantageous here that the load-bearing capacity that can be achieved in this manner is relatively low, in addition to which the snap-on connection thus formed leaves open a hollow space between the joint overlay and the tibia plateau when the prosthesis is in use.

Swiss Patent Specification CH 66 73 83 also describes a tibia part of a knee-joint prosthesis consisting of a tibia plateau with a journal and of a joint overlay. However, the installation of this knee-joint prosthesis requires individual preparatory work that is greatly dependent on the skill of the surgeon.

The invention has the objective of creating the possibility of configuring a knee-joint prosthesis of the above-mentioned type in such a way that the load-bearing capacity, especially the long-term load-bearing capacity, is considerably improved for a long period of time. Furthermore, a method for manufacturing such a knee-joint prosthesis is likewise to be provided.

The first objective is achieved according to the invention by means of a knee-joint prosthesis having the features of claim 1. Additional embodiments of the knee-joint prosthesis according to the invention can be gleaned from the subordinate claims 2 to 15.

Thus, according to the invention, an artificial joint is provided whose contact area or overdimension is configured differently, particularly as a function of the location, in such a way that, owing to the fixation forces that are exerted on the projection at the body temperature of the patient, a state of stress is created in the joint overlay and/or joint plateau that improves the load-bearing capacity and/or durability of the joint. In this context, the invention is based on the surprising realization that the thermal shrinkage connection achieved by the temperature difference and intended for the fixation of artificial joints can also be advantageously employed in order to enhance the load-bearing capacity of the joint by means of the fixation forces and the resultant stresses generated in this manner. For this purpose, the stresses are optimally selected in such a way that they counteract the forces exerted by loads. For instance, in this manner, a multi-dimensional state of stress can be created in which the joint overlay is only loaded by compression forces while the effect of tensile forces is ruled out by virtue of the pre-stressing.

For this purpose, according to a particularly advantageous embodiment of the invention, the fixation forces create a specific state of pressure that counteracts the external force applied under load.

Here, it is particularly practical for the contact area to be configured in such a way that the state of stress or the state of pressure can be adjusted in terms of its magnitude and/or direction so as to attain an optimal adjustment of the joint to the patient and to her/his individual requirements.

In this context, a variant that has proven to be especially strong is one in which the magnitude of the state of pressure brought to bear by the fixation forces is greater than the magnitude of the external force applied. As a result, only small tensile forces occur, even when large external forces are applied onto the joint overlay. In contrast, the load-bearing capacity with respect to external compression forces is increased, which goes hand in hand with an improvement of the load-bearing capacity.

An embodiment that is particularly relevant in actual practice is achieved when the state of pressure or state of stress can be adjusted as a function of the main load plane of the artificial joint. As a result, a state of stress that is adapted to the load that is exerted on the joint by the patient can already be achieved at the time when the joint overlay is affixed to the joint plateau. In this manner, owing to the thus optimized adaptation of the joint, the load limits can be further increased, particularly for generally known materials, without a need to alter the outer dimensions of the components in order to achieve this.

In an especially advantageous manner, it is achieved that the state of stress is prescribed by a pre-stressing that counters the external application of force.

The state of stress could already be largely established at the time of production. However, in order to be able to make an adaptation to the patient, in a particularly advantageous manner, an intermediate element for adjusting the state of stress is provided in the region of the contact area so that the state of stress can be variably adjusted depending on the location.

For example, for this purpose, the element can be affixed in different positions in order to ensure ease of adjustment by the surgeon.

A variant found to be particularly practical is one in which the element is configured as a bowl and which allows the joint plateau to be affixed at different angular positions with respect to the joint overlay.

For this purpose, an embodiment that has been found to be particularly advantageous is one in which the recess has an undercut that affixes the projection positively, said undercut being determined by the contour or topography of the joint overlay, especially by the thickness of the material since, as a result, the fixation forces that occur can be adjusted differently in various areas. Furthermore, varying overdimensions could be provided depending on the functional levels of the joint. Therefore, the state of stress in the joint can be considerably optimized, particularly in terms of the anticipated load, while concurrently prolonging the service life. In this context, the projection inserted into the recess is affixed positively and non-positively by means of the shrinkage connection. For this reason, a non-rotation-symmetrical, for example, kidney-shaped, joint overlay can be affixed without any limitations. For instance, a contact area that is slanted with respect to the joining direction generates a prestressing force that, at the same time, reliably rules out the possibility of a gap forming between the joint plateau and the joint overlay.

A particularly advantageous embodiment of the invention can also be achieved if the joint plateau and the joint overlay have a different coefficient of thermal expansion. As a result, warming or cooling the joint plateau together with the joint overlay causes a different heat expansion or cold shrinkage, so that a different size is established. Consequently, it is no longer necessary to exclusively warm up the recess and/or to cool off the projection and to join them in the state thus established.

In this context, it has been found to be particularly practical for the joint plateau and the joint overlay to be connected to each other in a way that their contours are flush with each other. This avoids undesired projections and the load-bearing capacity is considerably enhanced, even over a long period of time. Here, the outer dimensions, for example, the cross-sectional surface area of the joint plateau and of the joint overlay, match each other especially identically.

In contrast, a particularly simple variant of the knee-joint prosthesis according to the invention is one in which a contact area between the projection and the recess is imparted with surface characteristics that improve the force transmission such as, for example, especially roughness or structuring. This also considerably increases the load-bearing capacity of the connection owing to the improved static friction. For this purpose, the contact area is provided, for instance, with structuring.

A particularly practical embodiment of the invention is one in which a projection made of polyethylene is associated with the joint overlay and a recess made of metal is associated with the joint plateau. As a result, an optimal ratio of the coefficients of thermal expansion is achieved, so that a temperature difference of approximately 10° C. [18° F.] is already sufficient to cause an expansion difference of about 0.1 mm. This facilitates handling of the joint plateau and joint overlay during assembly, thus allowing a selection of the joint overlay that meets the individual requirements of the patient at the time of treatment.

It has also been found to be particularly advantageous if the projection and the recess lie against each other on an encircling and continuous contact area. This brings about a uniform introduction of force over the entire length of the contact area, as a result of which the load-bearing capacity of the connection can be further enhanced. Here, according to another particularly favorable variant of the present invention, the joint plateau has the recess while the joint overlay has the projection.

The second objective, namely, to provide a method for manufacturing an artificial joint, especially one configured as a knee-joint prosthesis, in which a joint plateau is connected to a joint overlay by means of a contact area formed by a recess and a projection inserted therein, is achieved according to the invention in that the contact area or overdimension is configured differently, particularly as a function of its location, in such a way that, owing to the fixation forces that are exerted on the projection at the body temperature of the patient, a state of stress is created in the joint overlay that improves the load-bearing capacity and/or durability. As a result, the fixation between the joint plateau and the joint overlay is realized in a manner that is simple to handle and that accounts for a considerably higher load-bearing capacity owing to the state of stress caused by the shrinkage connection.

The invention allows various embodiments. In order to better illustrate the basic principle, an embodiment is depicted in the drawing and described below. The drawing shows the following:

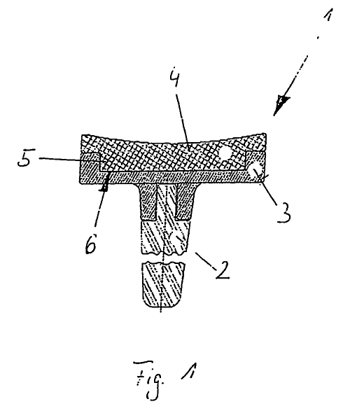

FIG. 1—a sectional side view of an artificial joint according to the invention.

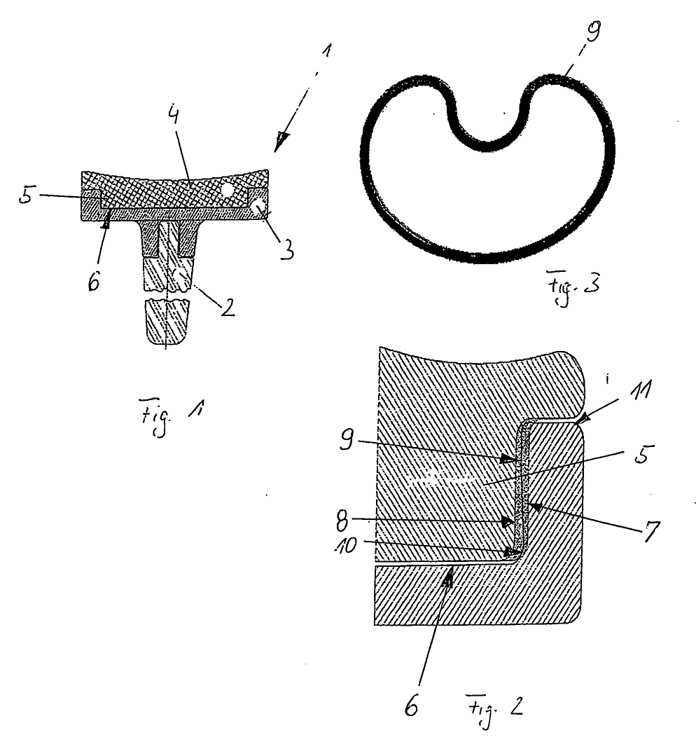

FIG. 2—a detailed depiction of the artificial joint shown in FIG. 1;

FIG. 3—a top view of the joint shown in FIG. 1.

FIG. 1 shows a sectional side view of an artificial joint 1, especially configured as a knee-joint prosthesis. This figure shows a joint plateau 3 fitted with a journal 2, said plateau being connected positively and non-positively to a joint overlay 4. For this purpose, the joint overlay 4 exhibits a projection 5 that has an overdimension 7 with respect to a recess 6 of the joint plateau 3. In order to be able to insert the projection 5 into the recess 6, first of all, a temperature is established that, when the projection 5 and/or the recess 6 is warmed up or cooled off, differs markedly from the body temperature of the patient, as a result of which different thermal expansions are obtained. Temperature equalization occurs after the projection 5 has been inserted into the recess 6, as a result of which the projection 5 is stressed in the recess 6 while concurrently coming to lie flat. The thermal shrinkage connection brought about by the temperature difference can concurrently be used advantageously to increase the load-bearing capacity of the joint I by means of the fixation forces applied in this manner and of the stresses thus generated. For this purpose, the stresses are optimally adjusted in such a way that they counteract the forces exerted as a function of the load. For example, a state of stress can be generated in this manner in which the joint overlay 4 is only stressed by compression forces while the application of tensile forces is ruled out by virtue of the pre-stressing.

The precise shaping of the projection 5 and of the recess 6 is better illustrated in FIG. 2, which depicts a detailed representation of the artificial joint 1 shown in FIG. 1. The ready-to-use functional position is shown in which the projection 5 is inserted positively and non-positively into the recess 6. The original overdimension 7 of the projection 5 (indicated by a broken line) is first reduced by cooling to the shrinkage size 8 (likewise indicated by a broken line), so that the assembly can be carried out without any problem. During the subsequent temperature equalization, the projection 5 is pressed flatly against the recess 6, where it becomes affixed non-positively. As a function of the slanted arrangement of a contact area 9 of the recess 6, an undercut 10 is formed which additionally brings about a positive fixation of the projection 5 in the recess 6. At the same time, this causes stress profiles that differ from each other in the area of the undercut 10 in comparison to an edge area 11 of the recess 6, so that the resulting force components cause a pre-stressing in the direction of the projection 5 with respect to the recess 6.

FIG. 3 shows a complementary top view of the joint 1 shown in FIG. 1. This figure shows the encircling contact area 9 between the projection 5 and the recess 6 shown in FIGS. 1 and 2. Here, the contact area 9 has a continuous course which ensures a uniform introduction of the fixation force over the entire circumference. In this way, the load-bearing capacity of the artificial joint I can be further increased.

1-16. (canceled)

17. An artificial joint for a patient comprising:
a joint plateau having a recess including a first dimension; and
a joint overlay having a projection corresponding to the recess and a second dimension, the second dimension being larger than the first dimension at a body temperature of the patient,
wherein the projection is insertable into the recess by virtue of a temperature difference of at least one of the projection and the recess with respect to the body temperature, a contact area being formed between the projection and the recess at the body temperature, and
wherein at least one of the second dimension and the contact area is determined such that fixation forces exerted on the projection at the body temperature create a state of stress in at least one of the joint overlay and the joint plateau, the state of stress improving at least one of a load-bearing capacity and a durability of the artificial joint.

18 The artificial joint as recited in claim 17, wherein a magnitude of the state of stress is adjustable in the joint overlay by the fixation forces.

19. The artificial joint as recited in claim 17, wherein the contact area is configured such that at least one of a magnitude and a direction of the state of stress is adjustable.

20. The artificial joint as recited in claim 17, wherein a magnitude of the state of stress is greater than a magnitude of the external force applied.

21. The artificial joint as recited in claim 17, wherein the state of stress is adjustable as a function of a main load plane of the artificial joint.

22. The artificial joint as recited in claim 17, wherein the state of stress is prescribed by a pre-stressing that counters an external application of force.

23. The artificial joint as recited in claim 17, further comprising an intermediate element disposed in a region of the contact area and configured to adjust the state of stress.

24. The artificial joint as recited in claim 23, wherein the intermediate element is affixable in different positions.

25. The artificial joint as recited in claim 23, wherein the intermediate element is configured as a bowl.

26. The artificial joint as recited in claim 17, wherein the recess has an undercut positively affixing the projection, the undercut being determined by at least one of a contour and a topography of the joint overlay.

27. The artificial joint as recited in claim 17, wherein the joint plateau and the joint overlay different coefficients of thermal expansion.

28. The artificial joint as recited in claim 17, wherein the joint plateau and the joint overlay are connected to each other in a way that contours of the joint plateau and the joint overlay are flush with each other.

29. The artificial joint as recited in claim 17, wherein a surface of at least one of the projection and the recess forming the contact area includes a surface characteristic for improving force transmission.

30. The artificial joint as recited in claim 29, wherein the surface characteristic includes at least one of a roughness and a structuring.

31. The artificial joint as recited in claim 17, wherein the projection includes a polyethylene and the joint plateau includes a metal.

32. The artificial joint as recited in claim 17, wherein the contact area encircles the projection and is continuous.

33. A method for connecting a joint plateau to a joint overlay of an artificial joint of a patient, the method comprising:
providing a joint overlay having a projection including a first dimension;
providing a joint plateau having a recess corresponding to the projection, the recess having second dimension larger than the first dimension at a body temperature of the patient;
bringing at least one of the projection and the recess to a temperature different from the body temperature;
inserting the projection into the recess; and
brining the projection and the recess to body temperature so as to form a contact area between the projection and the recess so as to connect the joint plateau to the joint overlay,
wherein the first dimension and the second dimension are determined such that fixation forces exerted on the projection at the body temperature create a state of stress in at least one of the joint overlay and the joint plateau that improves at least one of a load-bearing capacity and a durability of the artificial joint.
